(19) United States
(12) Patent Application Publication
(10) Pub. No.: US 2006/0105366 A1
(43) Pub. Date: May 18, 2006

(54) Specific base sequence detection method and primer extension reaction detection method

(75) Inventors: Shinichi Hiroshima, Yakohama-shi (JP); Hiroshi Takiguchi, Suwa-shi (JP); Hitoshi Fukushima, Suwa-shi (JP); Shinobu Yokokawa, Suwa-gune (JP)

Correspondence Address: OLIFF & BERRIDGE, PLC, P.O. BOX 19928, ALEXANDRIA, VA, 22320, US

(73) Assignee: Seiko Epson Corporation, Tokyo (JP)

(21) Appl. No.: 11/248,241

(22) Filed: Oct. 13, 2005

(30) Foreign Application Priority Data
Nov. 16, 2004 (JP) 2004-331367

(51) Int. Cl. C12Q 1/68

(52) U.S. Cl. 435/6

(57) ABSTRACT

A specific base sequence detection method, comprising preparing a sample solution including a target nucleic acid, a primer for amplifying a specific base sequence and whose end has a site to be coupled to an electrode, and nucleotide; extending the primer if the specific base sequence is present in the nucleic acid by putting the sample solution in a condition that causes an extension reaction of the primer; performing an electrical measurement by immersing an electrode in a measurement solution including the sample solution that has completed the extension reaction; and detecting whether the specific base sequence is present in the nucleic acid based on a result of the electrical measurement.

This application claims the benefit of Japanese Patent Application No. 2004-331367 filed on Nov. 16, 2004. The entire disclosure of the prior application is herby incorporated by reference herein its entirety.

### BACKGROUND

The present invention relates to a specific base sequence detection method and a primer extension reaction detection method.

Examining the presence of a nucleic acid that has a specific base sequence is a very important technology. It works as an integral part in diagnosing a genetic disease, testing food contamination with bacteria or viruses, and examining the human body for infections of bacteria or viruses, for example.

It becomes increasingly clear that some genetic diseases, such as severe combined immunodeficiency disease and familial hypercholesterolemia, are attributed to a specific genetic deficiency. Therefore, examining the presence of a gene that has a specific base sequence causing such diseases can be used for diagnostic purposes.

Food contamination caused by Escherichia coli O157, etc., has become a social problem in recent years. To test food for the presence of contaminants including bacteria and viruses, the presence of a base sequence of DNA or RNA specific to the suspected bacteria or viruses is examined. The same can be said for examining the human body for infections.

Detecting a specific base sequence in a nucleic acid requires high sensitivity, since a sample of the nucleic acid having this specific base sequence is usually small in amount. To increase detection sensitivity, for example, a polymerase chain reaction (PCR) method has been widely used for repeating primer extension reactions with a DNA polymerase to amplify a nucleic acid having a specific base sequence. Japanese Unexamined Patent Publication-No. 4-346800 is an example of related art.

Such a method for detecting a nucleic acid having a specific amplified base sequence, however, involves some problems. For example, one of the most versatile methods for detecting a nucleic acid having a specific amplified base sequence is electrophoresis. This method uses a carcinogen, e.g. ethidium bromide, as a fluorescent intercalator and thus requires careful handling. Furthermore, this electrophoresis method takes a long period of time for detection.

### SUMMARY

An advantage of the invention is to provide a technique for accurately detecting the presence of a specific base sequence in a target nucleic acid by a simple method.

A primer extension reaction detection method according to an aspect of the invention includes: preparing a sample solution including a target nucleic acid, a primer for amplifying a specific base sequence and whose end has a site to be coupled to an electrode, and nucleotide; extending the primer if the specific base sequence is present in the nucleic acid by putting the sample solution in a condition that causes an extension reaction of the primer; performing an electrical measurement by immersing an electrode in a measurement solution that includes the sample solution that has completed the extension reaction; and detecting whether the specific base sequence is present in the nucleic acid based on a result of the electrical measurement.

This method makes it possible to accurately detect whether the specific base sequence is present in the target nucleic acid by the electrical measurement (of impedance volume Z″, for example) with the sample solution that has completed the reaction. The method is applicable to tailor-made medicine, such as medication based on SNP typing.

Here, the primer may be composed of a complementary sequence that binds to the specific base sequence in a complementary manner. Also, a result of detecting whether the primer has been extended based on a result of the electrical measurement may be used to detect whether the specific base sequence is present.

The primer may include an upstream primer and a downstream primer at least one of whose end has the site to be coupled to an electrode.

The site to be coupled to an electrode may be either a thiol group, an amino group, or biotin. The electrical measurement may be either a measurement of impedance, current, or electrical charge of the electrode.

### BRIEF DESCRIPTION OF THE DRAWINGS

The invention will be described with reference to the accompanying drawings, wherein like numbers reference like elements, and wherein:

FIG. 1 is a diagram illustrating a PCR when a genomic DNA and each primer according to one embodiment of the invention are complementary;

FIG. 2 is a diagram illustrating a PCR when a genomic DNA and either of both primers according to the present embodiment are non-complementary;

FIG. 3 is a diagram illustrating an impedance measurement according to the present embodiment;

FIG. 4 is a diagram illustrating an impedance measurement according to the present embodiment; and

FIG. 5 is a chart showing measurement results of impedance volume Z″ according to the present embodiment.

### DETAILED DESCRIPTION OF EMBODIMENTS

This embodiment involves detection of the presence of a specific base sequence in a single nucleotide polymorphism (SNP) in a target DNA sample by means of a primer extension reaction (i.e. SNP typing). The SNP refers to a site having an altered base sequence that is present in one out of 1000 DNA sequences, and represents individual genetic characteristics including predisposition to diseases and sensitivity to medication.

This SNP typing starts with preparing a sample solution including a target genomic DNA having an SNP, a pair of upstream and downstream primers, Taq polymerase, a buffer, and dNTPs as follows. At the end of either the upstream or downstream primer included in this sample solution, a thiol group (a site to be coupled to an electrode) is attached. The description below assumes that a thiol group is attached to the end of the downstream primer.

Sample solution composition:

| | |
|---|---|
| dNTPs | (final concentration: 0.2 mM) |
| Upstream primer | (final concentration: 1.0 μM) |
| Downstream primer (20 bases) | (final concentration: 1.0 μM) |
| 10× buffer | (final concentration: 1× buffer) |
| Taq polymerase | (final concentration: 2 units) |
| Genomic DNA | (final concentration: 0.1 to 0.2 μg) |

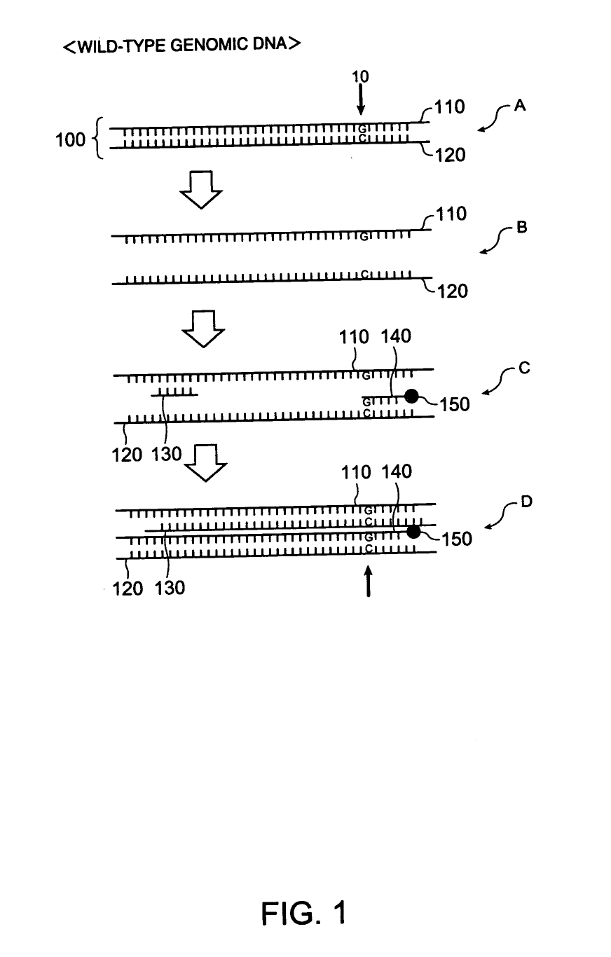

The prepared sample solution is put in the condition that causes a PCR (i.e. an extension reaction of each primer). FIG. 1 is a diagram illustrating a PCR when a genomic DNA and each primer are complementary (i.e. when using a wild-type genomic DNA). A PCR requires n cycles (e.g. 30 to 35 cycles) of the following three-step temperature changes. Specifically, a genomic DNA 100 having a target SNP 10 is thermally denatured by a first-step temperature change (up to 94 to 96 degrees Celsius, for example) for thermal denaturation, producing single-stranded DNAs 110, 120 (see FIGS. 1A and 1B). Of the single-stranded DNAs 110, 120, one DNA having genetic information is referred to as a target DNA 110, while another without genetic information is referred to as a complementary strand DNA 120.

Then by a second-step temperature change (down to 55 to 60 degrees Celsius, for example) for annealing, an upstream primer 130 is annealed to the target DNA 110, while a downstream primer 140 whose end is attached with a thiol group 150 is annealed to the complementary strand DNA 120 (see FIG. 1C). Subsequently, both the upstream primer 130 and the downstream primer 140 are extended by a third-step temperature change (up to 72 to 74 degrees Celsius, for example) for extension (see FIG. 1D). This process is repeated for n cycles to amplify both the target DNA 110 and the complementary strand DNA 120 2ⁿ-fold.

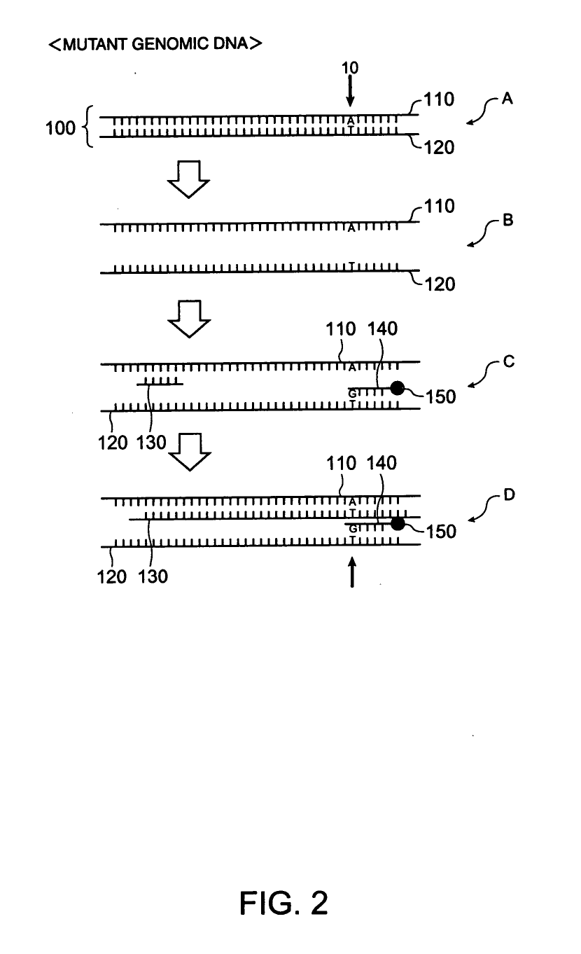

FIG. 2 is a diagram illustrating a PCR when a genomic DNA and at least either of the both primers are non-complementary (i.e. when using a mutant genomic DNA). While the description below of the present embodiment involves a case in which the genomic DNA and the downstream primer are non-complementary, the same can be said for another case in which the genomic DNA and the upstream primer are non-complementary.

In the same manner as mentioned above, the genomic DNA 100 having the target SNP 10 is thermally denatured by the first-step temperature change to produce the target DNA 110 and the complementary strand DNA 120 (see FIGS. 2A and 2B). Then by the second-step temperature change, the upstream primer 130 is annealed to the target DNA 110. The downstream primer 140, however, is not fully annealed to the complementary strand DNA 120 because of a mismatch at its end (between one base G of the downstream primer 140 and another base T of the complementary strand DNA 120 shown in FIG. 2C).

As a result of this annealing, the upstream primer 130 is extended while the downstream primer 140 is not by the third-step temperature change (see FIG. 2D). This process is repeated for n cycles to amplify the target DNA 110 2ⁿ-fold, while the complementary strand DNA 120 is not amplified.

Consequently, an extension reaction occurs when the primer is composed of a complementary sequence that binds to a specific base sequence in a complementary manner. Meanwhile, if the primer is composed of not such a complementary sequence but a non-complementary sequence that does not bind to a specific base sequence in a complementary manner, no extension reaction extending the chain length occurs.

Following the above-described cycles to complete the PCR, the sample solution that has completed the PCR is placed in the measurement solution below to start an impedance measurement.

Measurement solution composition:

| | |
|---|---|
| PBS (pH 7.0) | (50 mM) |
| NaCl | (1 M) |
| MgCl₂ | (10 mM) |

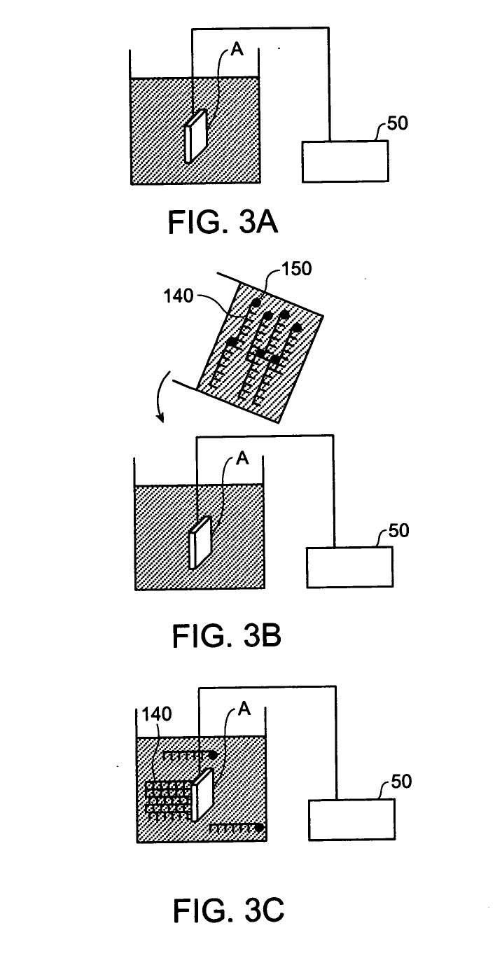

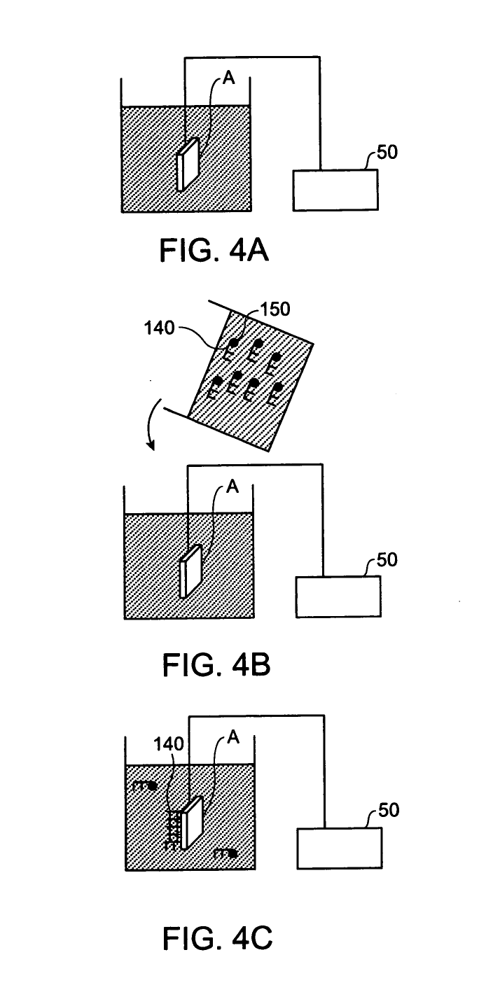

FIG. 3 illustrates an impedance measurement with one sample solution that has caused an extension reaction (referred to as the “reacted sample solution”). FIG. 4 illustrates an impedance measurement with another sample solution that has not caused an extension reaction (referred to as the “unreacted sample solution”).

After preparing a 10 ml of the measurement solution, an electrode substrate (a gold electrode substrate with an electrode area of about 3 mm diameter according to the present embodiment) is immersed in the measurement solution for about five minutes. Then impedance volume Z″ (imaginary part) is measured with an impedance measuring device 50 coupled to this electrode substrate A as shown in FIGS. 3A and 4A. After 500 seconds of the measurement, each sample solution (1 μM, 100 μl) is poured into the measurement solution as shown in FIGS. 3B and 4B. The impedance volume Z″ is measured once in ten seconds at 100 Hz until 3000 seconds have passed.

As mentioned above, each sample solution includes a great amount of the downstream primer 140 whose end is attached with the thiol group 150. This group 150 serves to fix the downstream primer 140 onto the surface of the electrode substrate A. Specifically, the downstream primer 140 whose chain length has been extended is fixed onto the surface of the electrode substrate A in the reacted sample solution as shown in FIG. 3C, while the downstream primer 140 whose chain length has not been extended is fixed onto the surface of the electrode substrate A in the unreacted sample solution as shown in FIG. 4C.

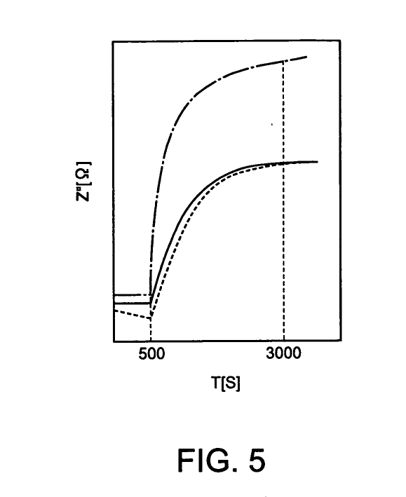

FIG. 5 is a chart showing measurement results of the impedance volume Z″. In this chart, the dashed line represents measurement results with the extension reaction, while the dotted line represents measurement results without the extension reaction. For comparison, the thick solid line represents measurement results of the impedance volume Z″ in a comparison test with a probe having an oligo DNA with a 20-base chain length fixed to an electrode substrate under the same condition as described above.

Referring to FIG. 5, the impedance volume Z″ differed greatly between the cases with and without the extension reaction. Specifically, one case without the extension reaction showed nearly the same results as the comparison test (compare the dotted and thick solid lines in FIG. 5), while another case with the extension reaction showed greatly different results from the comparison test (compare the dashed and thick solid lines in FIG. 5). By comparing the impedance volume Z″ in this way, it is possible to accurately detect whether the extension reaction has occurred (or detect whether a specific base sequence is present). Note that multiple different types of probes (shown below) having 20 bases with different compositions were prepared to measure the impedance volume Z″ under the same condition as described above in the comparison test, and there were no significant differences in their measurement results. This means that similar results (impedance volume Z″) can be given from different probe compositions (base sequences) as long as the probes have the same number of bases. Also, the number of bases is not limited to 20, and can be 5 or 49, for example.

Probe composition (20 bases):

5′HS-C₆H₁₂-AAAAAAAAAAAAAAAAAAAA 3′
5′HS-C₆H₁₂-TTTTTTTTTTTTTTTTTTTT 3′
5′HS-C₆H₁₂-GGGGGGGGGGGGGGGGGGGG 3′
5′HS-C₆H₁₂-CCACACTCACAGTTTTCACT 3′
5′HS-C₆H₁₂-TTTTCACTTCAGTGTATGCG 3′

According to the method that has been described, it is possible to accurately detect whether the extension reaction has occurred (or detect whether a specific base sequence is present in an SNP in the present embodiment) with the simple measurement of the impedance volume Z″. Therefore, this method is applicable to tailor-made medicine, such as medication based on SNP typing.

While a DNA having an SNP is used as the target DNA in the present embodiment, a DNA extracted from a zoograft, fungus, cultured cell or the like and having no SNP can also be used as the target. In this manner, this method is applicable to diagnosing a genetic disease, testing food for the presence of contaminants including bacteria and viruses, and examining the human body for infections of bacteria and viruses.

While a gold electrode substrate is used as the electrode substrate in the present embodiment, an electrode made of other metal materials can be used instead. In this case, a functional group (a site to be coupled to an electrode), such as an amino group or biotin, that is required for fixation depending on the type or the like of the electrode substrate may be attached to a primer end.

While the method according to the present embodiment measures the impedance volume Z″ (electric measurement) to detect whether the extension reaction has occurred (or detect whether a specific base sequence is present in an SNP), it is also possible to compare not the impedance volume, but the amount of current by a current measurement (electric measurement) or the quantity of electrical charge by a charge measurement (electric measurement) in order to detect whether the extension reaction has occurred. It is also possible to introduce fluorescent molecules in a sample during a PCR and observe fluorescence in order to detect whether the extension reaction has occurred.

While the method according to the present embodiment uses a primer composed of a complementary sequence that binds to a specific base sequence in a complementary manner, the invention is also applicable to a primer partly including a non-complementary sequence, such as Allele Specific Primer (ASP) developed by Toyobo Co., Ltd. The ASP is designed as the second base from the 3′ end of the primer corresponds to an SNP and the third base from the 3′ end is always non-complementary to a target base. By attaching a site to be coupled to an electrode to the end of this ASP, it is possible to detect whether the extension reaction has occurred without complicated processing.

What is claimed is:

1. A specific base sequence detection method, comprising:
preparing a sample solution including a target nucleic acid, a primer for amplifying a specific base sequence and whose end has a site to be coupled to an electrode, and nucleotide;
extending the primer if the specific base sequence is present in the nucleic acid by putting the sample solution in a condition that causes an extension reaction of the primer;
performing an electrical measurement by immersing an electrode in a measurement solution including the sample solution that has completed the extension reaction; and
detecting whether the specific base sequence is present in the nucleic acid based on a result of the electrical measurement.

2. The specific base sequence detection method according to claim 1, the primer being composed of a complementary sequence that binds to the specific base sequence in a complementary manner.

3. The specific base sequence detection method according to claim 1, a result of detecting whether the primer has been extended based on a result of the electrical measurement being used to detect whether the specific base sequence is present.

4. The specific base sequence detection method according to claim 1, the primer including an upstream primer and a downstream primer at least one of whose end has the site to be coupled to an electrode.

5. The specific base sequence detection method according to claim 1, the site to be coupled to an electrode being one of a thiol group, an amino group, and biotin.

6. The specific base sequence detection method according to claim 1, the electrical measurement being one of a measurement of impedance, current, and electrical charge of the electrode.

7. A primer extension reaction detection method, comprising:
preparing a sample solution including a target nucleic acid, a primer for amplifying a specific base sequence and whose end has a site to be coupled to an electrode, and nucleotide;
extending the primer if the specific base sequence is present in the nucleic acid by putting the sample solution in a condition that causes an extension reaction of the primer;
performing an electrical measurement by immersing an electrode in a measurement solution that includes the sample solution that has completed the extension reaction; and
detecting whether the primer has been extended based on a result of the electrical measurement.


SEQUENCE LISTING (5 sequences)

SEQ ID NO: 1 - DNA, 20 bp, artificial sequence
  a probe immobilized on an electrode
aaaaaaaaaaaaaaaaaaaa

SEQ ID NO: 2 - DNA, 20 bp, artificial sequence
  a probe immobilized on an electrode
tttttttttttttttttttt

SEQ ID NO: 3 - DNA, 20 bp, artificial sequence
  a probe immobilized on an electrode
gggggggggggggggggggg

SEQ ID NO: 4 - DNA, 20 bp, Homo sapiens
ccacactcacagttttcact

SEQ ID NO: 5 - DNA, 20 bp, Homo sapiens
ttttcacttcagtgtatgcg